(19) United States
(12) Patent Application Publication
(10) Pub. No.: US 2020/0129229 A1
(43) Pub. Date: Apr. 30, 2020

(54) VENOUS ABLATION CATHETER AND METHOD

(71) Applicant: Anil K Chagarlamudi, Houma, LA (US)

(72) Inventor: Anil K Chagarlamudi, Houma, LA (US)

(21) Appl. No.: 16/460,858

(22) Filed: Jul. 2, 2019

Related U.S. Application Data
(60) Provisional application No. 62/693,223, filed on Jul. 2, 2018.

(51) Int. Cl. A61B 18/14; A61M 25/06

(52) U.S. Cl. CPC A61B 18/1492; A61B 2018/00577; A61M 25/0606

(57) ABSTRACT

An improved venous ablation catheter and method of use. More particularly, the present invention relates to a radio-frequency ablation catheter that ablates and injects tumescent from inside a vein simultaneously. The catheter device comprises a distal end, proximal end, a needle and lumen for the needle to pass through, and an interior housing. After the vein to be ablated is cannulated and the catheter device is advanced into the vein, the needle is advanced through the venous wall into the peri-venous sheath and tumescent fluid or other fluid or gas is injected around the vein through the needle. The vein segment that has been injected with tumescent is sequentially ablated with the catheter device, and the catheter device is then drawn a distance out of the vein. At this point, the needle can be advanced through the venous wall again injecting tumescent or other fluid. This process can be repeated until the treatment is completed without having to remove the catheter device from the vein, thus requiring only one needle stick and fewer office visits for the patient.

### CROSS-REFERENCE TO RELATED APPLICATIONS

This application claims priority to U.S. Provisional Application No. 62/693,223 filed on Jul. 2, 2018, the contents of which are incorporated herein by reference in its entirety.

### FIELD OF THE INVENTION

The present invention is in the field of ablation and related to delivering fluid around blood vessels from inside the lumen of the vessels. Specifically, the present invention is related to an improved ablation catheter that ablates and injects tumescent from inside a vessel simultaneously.

### BACKGROUND

Venous ablation therapy is a well-known procedure. Chronic venous insufficiency (CVI) is the reflux of blood in the veins of lower extremities due to incompetent valves. CVI is very prevalent in the general population and affects 30-50 million people in United States. In CVI, the blood pools in the lower legs causing symptoms including, but not limited to, heaviness, burning, aching, restless legs, hyperpigmentation, and edema. In severe cases, CVI can cause skin necrosis, varicose veins, ulcers and wounds. Varicose veins can get thrombosed causing extreme pain or can rupture causing severe bleeding. Thus, symptomatic CVI must be treated.

Ligation and stripping was the prior standard of care. For the past three decades, ablation of incompetent veins has been the most preferred treatment of choice. This is performed with heat generated by radio frequency or laser energy or other heating element and entails insertion of a radiofrequency or laser catheter into the incompetent vein and ablating it. The ablation catheter reaches a very high temperature resulting in denaturation of proteins and vessel closure. Specifically, in the ablatio procedure, the ablation catheter reaches a temperature of 120 degrees C., and tumescent fluid is injected into the peri-venous sheath around the vein to be ablated to absorb heat that is severe enough to cause extreme discomfort. The high heat generated causes severe pain and can result in tissue injury. Accordingly, normal saline along with lidocaine and other agents is injected around the vein to absorb the heat generated. This fluid also compresses the vein around the ablation device causing better contact and more efficient transfer of heat energy. The ablation procedure is achieved by a series of transcutaneous needle sticks along the course of the target vein. The numerous needle sticks can be painful and leads to some patients failing to return for a second procedure, if needed.

The current method of injecting fluid around a vein takes a certain level of skill and finesse, but the current ablation procedures known in the art can be imprecise. The current ablation procedures are even more difficult in obese patients or if the vein is deep seated. The transcutaneous needle utilized in the procedure can also injure tissue, blood vessels and or nerves along its course causing bleeding, hematomas, and numbness. Also, the multiple needle sticks can transmit bacteria and infection from the skin into deep tissues. Many patients have open wounds making the ablation procedure riskier and more difficult, while other patients prefer to receive anxiolytics and or sedatives prior to procedure which may have side effects. Therefore, a need exists for an improved ablation catheter that requires less needle sticks to the patient.

The following references are incorporated herein by reference: Tim A. FISCHELL, M D, et al., Transcatheter Alcohol-Mediated Perivascular Renal Denervation With the Peregrine System, JACC Cardiovascular Interventions, VOL. 9, No. 6, 2016; available at: http://dx. doi. org/10.10.16/j j cin.2015.11. 041; US Patent Publication Nos. 2013/0030410, 2016/0184011, and 2018/0110561; U.S. Pat. Nos. 8,365,741, 7,644,715, 7,087,052, and 5,496,267; and International Patent Application No. WO 2018/056940.

### SUMMARY OF THE INVENTION

We disclose herein an improved catheter device and method of venous ablation requiring fewer needle sticks in the patient wherein tumescent or other fluid or gas is injected from the inside of the vein into the peri-venous sheath via a needle that exits the ablation catheter and injects fluid precisely around the vein. The catheter device is capable of ablating and injecting tumescent in adjacent segments of the vein simultaneously.

The catheter device comprises a distal end, proximal end, a needle and lumen for the needle to pass through, and an interior housing. After the vein to be ablated is cannulated and the catheter device is advanced into the vein, the needle is advanced through the venous wall into the peri-venous sheath and tumescent fluid or other fluid or gas is injected around the vein through the needle. The vein segment that has been injected with tumescent is sequentially ablated with the catheter device, and the catheter device is then drawn a distance out of the vein. At this point in the procedure, the needle can be advanced through the venous wall again injecting tumescent. This process be repeated until the treatment is completed without having to remove the catheter device, thus requiring as few as one needle stick and fewer office visits for the patient.

### BRIEF DESCRIPTION OF THE DRAWINGS

Further advantages of the invention will become apparent by reference to the detailed description of an embodiment of the invention when considered in conjunction with the drawings which form a portion of the disclosure and wherein:

FIG. 1 is a perspective view of an embodiment of the catheter device.

FIG. 2 is a perspective view of an embodiment of the catheter device.

FIG. 3 is a perspective view of an embodiment of the catheter device.

FIG. 4 is a perspective view of an embodiment of the catheter device.

FIG. 5 is a perspective view of an embodiment of the catheter device.

FIG. 6 is a perspective view of an embodiment of the catheter device.

FIG. 7 is a perspective view of an embodiment of the catheter device.

FIG. 8 is a perspective view of an embodiment of the catheter device.

### DETAILED DESCRIPTION

The following detailed description is presented to enable any person skilled in the art to make and use the invention. For purposes of explanation, specific details are set forth to provide a thorough understanding of the present invention. However, it will be apparent to one skilled in the art that these specific details are not required to practice the invention. Descriptions of specific applications are provided only as representative examples. Various modifications to the preferred embodiments will be readily apparent to one skilled in the art, and the general principles defined herein may be applied to other embodiments and applications without departing from the scope of the invention. The present invention is not intended to be limited to the embodiments shown but is to be accorded the widest possible scope consistent with the principles and features disclosed herein.

The current invention is an improved method of treatment of incompetent saphenous veins through ablation to alleviate symptoms of CVI. The catheter device of the present invention provides an improved method of radiofrequency where the tumescence or other fluid or gas is injected from the inside of the vein into the peri-venous sheath via a needle that exits the ablation catheter and injects fluid precisely around the vein. The depth of penetration of the needle is controlled by the operator by visualizing it under ultrasound. The tumescent is injected just before ablation, so it is cooler than body temperature and can absorb more heat reducing amount of tumescent fluid needed. This reduces the extravasation of injected fluid and the possibility of skin blistering.

The catheter ablates and injects tumescent in adjacent segments simultaneously, thus reducing the procedural time significantly. More importantly, there are no needle sticks for the tumescent making this step completely pain-free. The risk of infection is also lessened significantly because the skin is not punctured for the tumescence. Additionally, since the additional locations of the skin are not punctured, a large area of skin does not need to be prepped decreasing set up time and turn-over time between procedures. Thus, the apparatus and method of the present invention saves time, material, and labor. This procedure can be performed safely and with minimal to no pain for the patient even by less experienced operators. It also decreases the chance of infections and skin blistering.

The catheter device 10 of the present invention is designed to ablate and inject tumescent 43 or other liquid or gas from inside a vein simultaneously. The catheter device 10 and method may be used, for example, to treat venous insufficiency. In a preferred method, the catheter device 10 ablates and injects tumescent 43 simultaneously. Preferably, the tumescent 43 is injected into the peri-venous sheath 14, thus avoiding multiple needle sticks on the skin significantly reducing pain and infection.

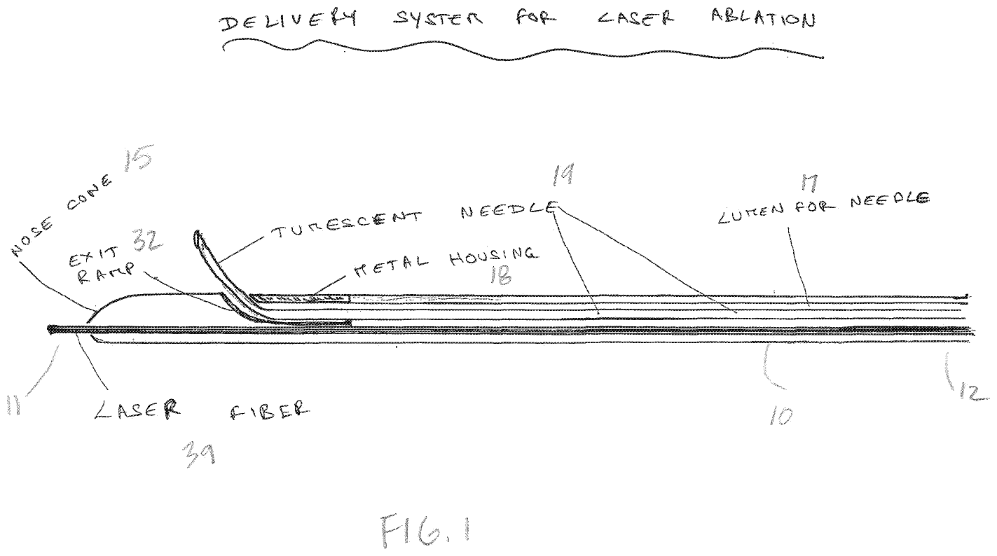

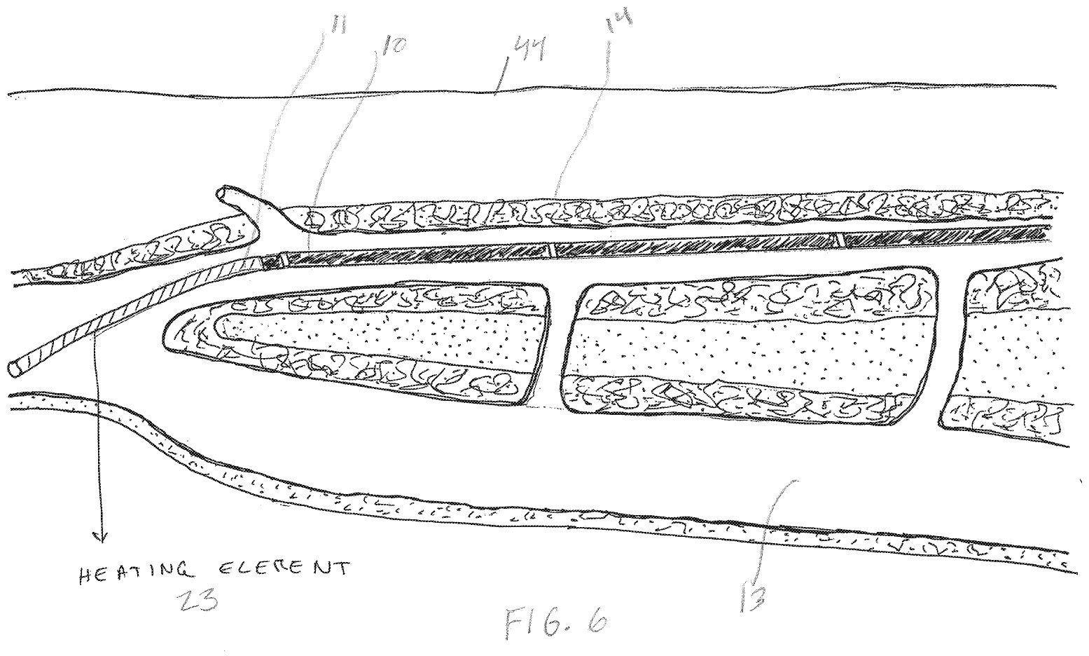

The catheter device 10 further comprises a distal end 11, proximal end 12, a needle 19 and lumen 17 for the needle 19 to pass through, and a housing 18, as shown in FIG. 1. The catheter device may further comprise a heating element, as shown in FIG. 6.

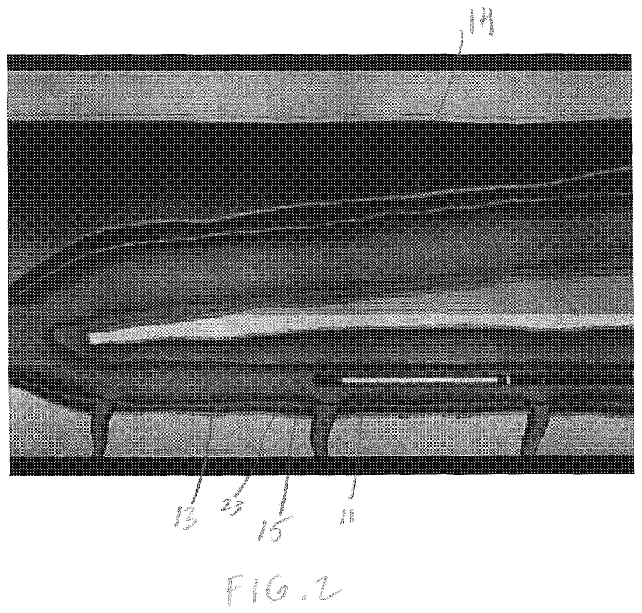

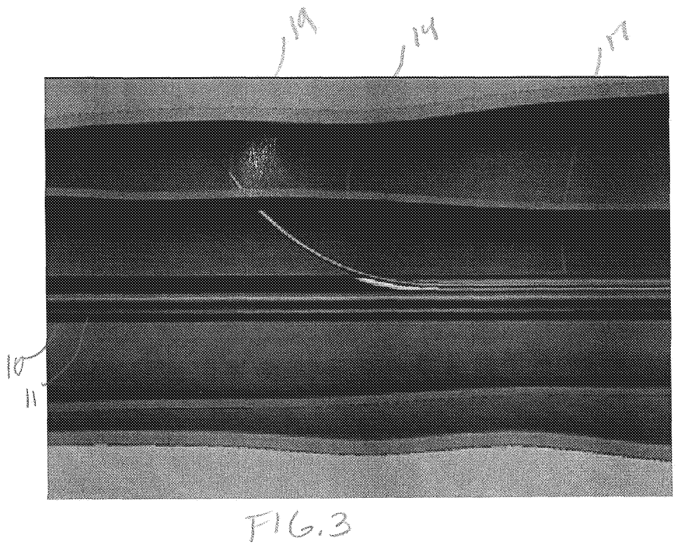

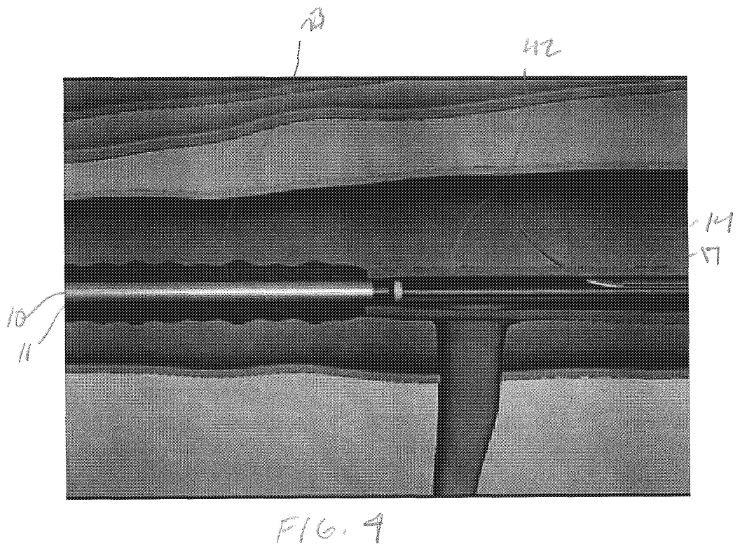

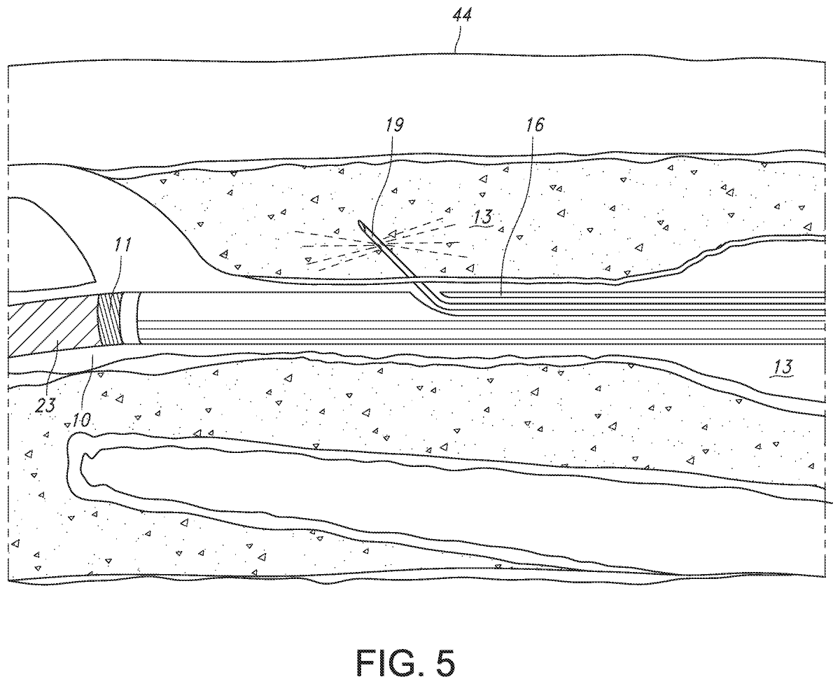

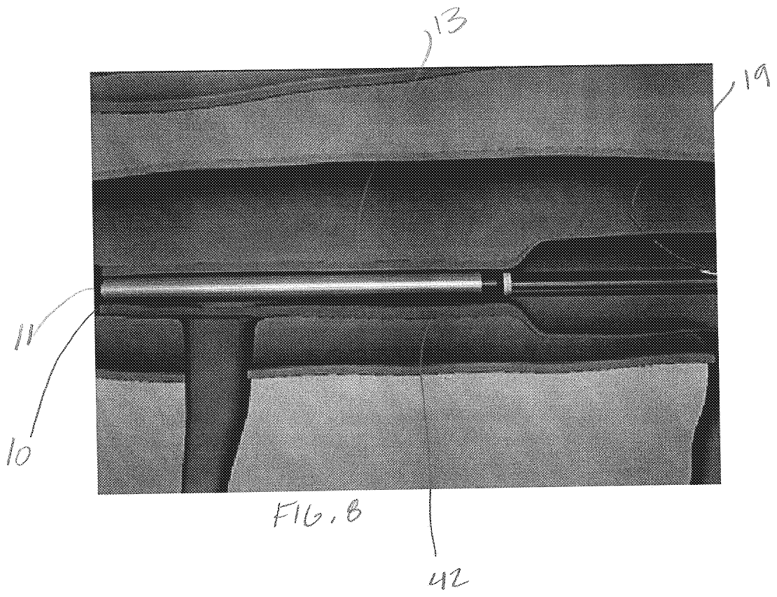

The present invention also includes a method of using the catheter device 10 for venous ablation procedures, such as those used to treat venous insufficiency. In an embodiment of the method, first, after being inserted through the skin 44 of a patient, the vein cannulating the vein 13 to be ablated is cannulated. Then the catheter device 10 is advanced into the vein 13, as shown in FIG. 2. Then, the needle 19 is advanced through the venous wall 16 into the peri-venous sheath 14, as shown in FIG. 3. Tumescent 43 is injected around the vein 13 through the needle 19 that has advanced 15 into the peri-venous sheath 14, as shown in FIG. 5. The vein 13 segment that has been injected with tumescent 43 is sequentially ablated with the catheter device 104, as shown in FIG. 4, causing the tumesced portion 42 of the vein 13 to collapse around the catheter device 10, as shown in FIG. 8. Finally, the catheter device 10 is then drawn a distance out of the vein 13. At this point, the needle 19 can then be advanced through the venous wall 16 again injecting tumescent 43, as shown in FIG. 5 and can be repeated until the treatment is completed without having to remove the catheter device 10.

After cannulation of the vein 13 to be treated at the location that is determined at preoperative evaluation, a peri-venous sheath 14 is inserted in the vein 13 and flushed. The ablation catheter 10 is then inserted via the sheath and advanced beyond the saphenofemoral or saphenopopliteal junction by visualizing under ultrasound. The ablation catheter may also be inserted by itself without the sheath. The catheter is flushed and patient is placed in Trendelenburg position or the leg is elevated to empty the vein.

The catheter device 10 is a slender, flexible tube or guide that has a distal or working end 11 and a proximal end 12. The distal end 11 is inserted into the vein 13 to be treated at a site determined based on the location of the venous reflux and symptoms.

It is contemplated herein that the catheter device 10 can be of varying lengths and have a single lumen 17 or multiple lumens. All the lumens can be of the same or of varying diameter. The catheter device 10 can be of varying diameter, for example 3F to 12 F, but could be of other sizes also.

In an embodiment of the invention, there are markings on the surface of the catheter device 10 at varying or regular intervals. These markings can encircle a variable portion of the circumference or can be circumferential. The markings can be alphanumerical, signs, symbols, lines or shapes or patterns of any kind. The markings can all be of one category or a combination of the above. There may also be words, abbreviations or logos also on the catheter.

The material used for the catheter device 10 can be of any material or polymer or metal or alloy or any combination of different materials.

The distal end 11, also referred to as the working end, of the catheter device 10 can have a blunt end or be round or have a nose cone of varying tapers. If tapered, the tapered portion may start at any length from the distal end 11 of the catheter device 10. The tip 15 may be of the same color or of a different color than the catheter device 10. The tip 15 of the catheter device 10 may be of the same material or of a different material.

The distal end 11 of the catheter device 10 can have a heating element 23 of varying lengths anywhere from 1 cm to 30 cm. The heating element 23 may include, but is not limited to, heating coils, a laser 39 or radiofrequency. This is the portion of the catheter device 10 that is in contact with the venous wall 16 and results in ablating the vein 13 with heat or other energy resulting in vessel closure and hence causing no flow in the lumen 17.

In an embodiment of the invention, the wires or filaments 24 in the heating element 23 at the proximal end 12 may run in a linear fashion along the length of the catheter device 10 or can be in a spiral, helical or other pattern. These wires 24 extend from the heating element 23 to the proximal end 12 of the catheter device 10 and into the handle 25 and from there are connected to an energy delivering device by a power cable 26. Energy may also be delivered without any wires 24 via other modes.

The wires 24 from the heating element 23 to the handle 25 may run in the wall 27 of the catheter device 10 or may run in a designated lumen by themselves or along with other structures. The wires 24 may or may not be coated with a non-conductive material to act as an insulation. The wires 24 can be of the same or varying diameter. The wires 24 can be round, oval or other shapes in cross section. The material used for the heating coil 23 and the wires 24 can be of any of stainless steel, nickel, chromium, ferrous, titanium, or any alloy.

There may be one or multiple thermistor(s) or temperature sensor(s) 28 at any distance from the distal end 11 of the catheter device 10. The wire or wires 29 from this/these thermistor(s) 18 run along the length of the catheter device 10 to the handle 25 at the proximal end 12. If more than one of these are present, these can be at variable distances from the distal end of the catheter device 10. This/these wires 29 are eventually connected to the energy generating device or console and provide a temperature readback, which is important to decide if adequate tumescent 43 or cooling agent has been delivered around the heating element 23 prior to starting the ablation.

There may be an opening 30 at the distal end 11 of the catheter device 10 from which a lumen 17 runs along the length of the catheter device 10 up to the proximal end 12 of the handle 25. The lumen 17 may also exit on the side of the catheter device 10 at any distance from the distal end 11 and not reach the handle 25. The lumen 18 can be of any diameter to allow passage of guide wires 31 of varying diameters. This lumen 17 may need to be flushed prior to insertion.

There is a housing 18 inside the catheter device 10 proximal to the heating element 23. This can be at varying distances from the heating element 23. This has a curved or angled ramp 32 inside that communicates to the side of the housing 18 on a proximal end 33 and to a hollow core 35 on the distal end 34. This is to facilitate exit of a needle 19. The housing 18 may have a closed end beyond the ramp 32 or there may be an opening in the ramp that communicates to the distal end 34 of the housing 18. This is to allow passage of a guide wire 31 from one end to the other through the housing 18.

The housing 18 may be made of stainless steel, titanium, nickel, ferrous, or any metal or alloy. This can be of any diameter and may allow passage of a needle 19 or guide wires 24 through it.

The needle 19 used to deliver tumescent 43 or gas is inside the housing 18 when in the retracted position. When advanced, the needle 19 rides the ramp 32 and exits the catheter device 10 and pierces the venous wall 16 and enters the space around the vein 13. The needle 19 pierces the vessel wall 16 at an angle or near perpendicular or perpendicular fashion.

The distance that the needle 19 exits the venous wall 16 is visualized by ultrasound or other modes.

The depth of penetration can be controlled by the operator. There may be an auto-stop mechanism that can be set to any desired depth of penetration.

The needle 19 is retracted into the housing 18 in the catheter device 10 either manually, or by a spring, or other mechanisms when released.

The needle 19 is made of nitinol or other metal or alloy or a suitable plastic or plastic polymer or any other suitable material or compound. The needle 19 can be of varying sizes from gauge 30 to 12 gauge. The needle can be of any curvature. The needle may also be echogenic to facilitate visibility on ultrasound.

The end of the needle 19 that pierces the venous wall 16 may be beveled. This bevel can be of any suitable angle.

The exit port 36 for the fluid or gas may be at the end of the needle 19 in the beveled portion 37. There may be additional ports for fluid or gas delivery on the needle shaft 38 of the needle 19 at varying distances from the end. There may be single or multiple ports on the needle shaft 38 and these may all face in one direction or in several directions along the shaft 38. If there are any ports on the shaft 38 of the needle 19, the bevel 37 may have an open port or this can be closed.

The bevel 37 faces forward or is facing the distal end 11 of the catheter device 10 when advanced out of the catheter device 10. This is to facilitate easy riding on the housing ramp 32 and to avoid friction between the pointed end and the ramp 32 if advanced the opposite way.

The needle 19 continues proximally inside the catheter device 10 in a designated lumen 17 by itself or along with other structures. The needle 19 continues into the handle 25 and exits the handle 25. There is a luer-lock or other connecting mechanism at the end of the needle 19 that connects to the fluid or gas tubing through which the tumescent 43 or other liquid or gas flows.

Fluid or gas can be infused either manually, with the help of an infusion pump, pressure bag or other methods through the needle 19.

In an embodiment of the invention, the fluid is a combination of saline, lidocaine or other anesthetic agent and a pH modifying agent like Sodium bicarbonate. This fluid is injected via the needle 19 after the needle 19 is advanced into the space outside the vein 13. This fluid compresses the vein 13 from the outside and results in better contact of the inside of the venous wall 16 with the heating element 23. This facilitates conduction of the thermal energy more efficiently.

The heat delivered to the venous wall 16 can be very high resulting in severe pain. To alleviate this the fluid around the vein 13 absorbs the heat and acts a heat sink.

The catheter may have a balloon at the proximal end 12, a short distance before the heating element 12. When collapsed the balloon hugs the catheter device 10 and may or may not be seen as a slight bulge on the catheter device itself 10. This balloon may be cylindrical, oval shaped, fusiform, or of any other shape.

This balloon is in communication with the handle 25 via a lumen 17 that runs in the catheter device 10. The balloon may be partially or fully inflated with a syringe attached to the distal end 12 by a connecting mechanism.

The balloon can be of any capacity of up to 8 ml. The balloon when inflated fully can reach a diameter of between 2-25 mms.

When ablation is performed there could be retained blood in the vein 13 around the heating element 23. This forms a coagulum post procedure and can result in pain or cause phlebitis. Hence the balloon could be inflated up to the desired diameter so as to partially or fully occlude the lumen 17 and retracting the catheter device 10 in this fashion exsanguinates the vein 13 being ablated resulting in a dry vein. This results in no or minimal coagulum which decreases post procedural pain. This eliminates a thrombectomy procedure that might be needed otherwise.

In an alternate embodiment of the invention, the distal end 11 of the catheter device 10 may not have a heating element as previously described. This modification is used when a non-radiofrequency mode, such as a laser, is used. In this modification, the end of the laser fiber 39 exits the catheter device 10 via the distal lumen 17 up to a desired distance.

In this embodiment, fluid is injected prior to pulling the laser fiber 39 back. The end of the laser fiber 39 can be pulled back any amount of distance while ablating as long as there is enough tumescent 43 surrounding the vein 13 to absorb the heat.

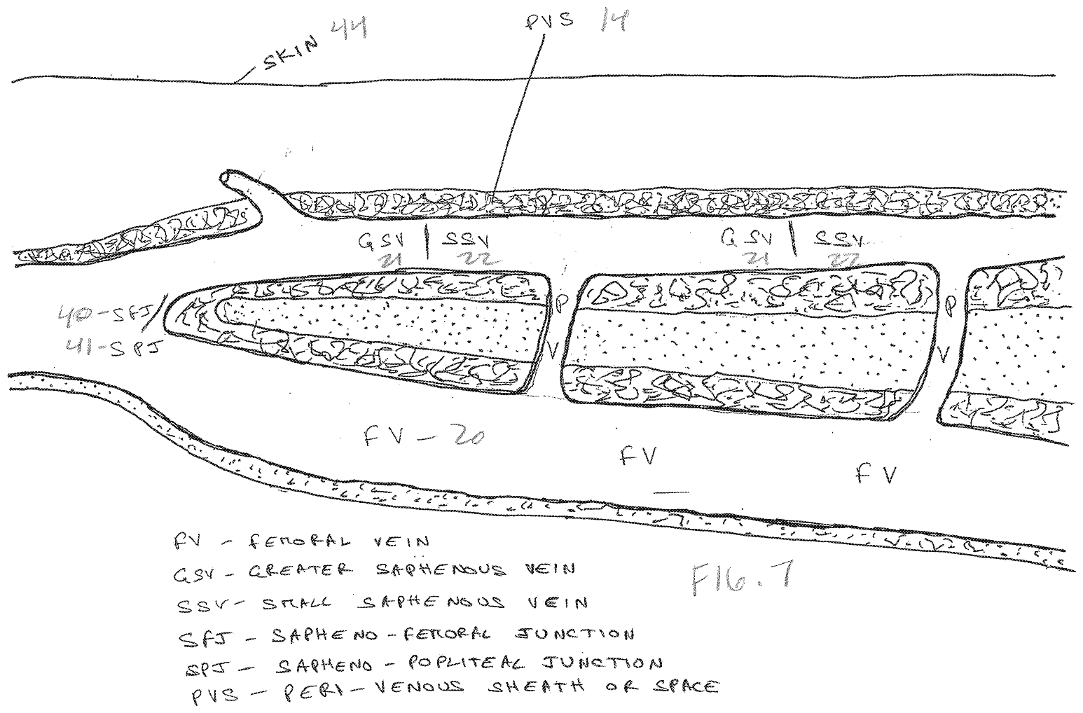

In an exemplary embodiment of using the catheter device 10 in a venous ablation procedure, a saphenous vein 13 is treated, as shown in FIG. 7. Specifically, the catheter device 10 is advanced through the femoral vein 20 beyond the sapheno-femoral 40 or saphenopopliteal junction 41 into the greater saphenous vein 21 and small saphenous vein 22. The needle 19 is advanced into the peri-venous sheath 14 to inject tumescent 43. The tumescence is completed causing the tumesced portion 42 of the vein 13 to collapse around the catheter device 10, as shown in FIG. 3. The catheter device 10 is then withdrawn a distance while remaining in the general location of the greater saphenous vein 21 and small saphenous vein 22, and tumescent 43 is injected into the next segment of the vein 13 being treated, as shown in FIG. 5.

In an embodiment of the invention, the tumescent needle 19 that is inside the catheter device 10 is advanced by a mechanism on the handle 25. The needle 19 then pierces the venous wall 16 and enters the space around the vein 13. Pressure can be applied with the ultrasound probe while visualizing this. This may also facilitate the needle 19 to puncture the vein 13. Tumescent fluid 43 is then injected into the perivenous space to inundate the vein 13 to be ablated.

The needle 19 is then withdrawn into the catheter device 10, and the catheter device 10 is retreated by a distance that is approximately equal to the length of the heating element 23. The ablation is then started. While the vein 13 is being ablated, the needle 19 is advanced in the preceeding segment. After ablation of the first segment is completed the catheter device 10 is retreated to the desired length and the process is repeated.

There is no percutaneous needle sticks. This reduces chance of infection and risk of needle stick injury to the personnel. Tumescent 43 can be injected precisely around the vein 13 with much ease. After the last segment is ablated the catheter is removed from the body and compression dressing applied.

Although the specification speaks to veins, it is contemplated herein that the catheter device 10 is capable of being used in all vessels.

Additionally, although the specification teaches tumescent 43 as being injected by the needle 19 of the catheter device 10, any liquid or gas capable of being injected into a vessel wall or outside of a vessel is contemplated herein.

An embodiment of the catheter device 10 includes a control mechanism to advance and retract the needle 19.

Another embodiment of the catheter device 10 includes an auto-retract feature to retract the needle 19 during the ablation procedure.

The terms “comprising,” “including,” and “having,” as used in the claims and specification herein, shall be considered as indicating an open group that may include other elements not specified. The terms “a,” “an,” and the singular forms of words shall be taken to include the plural form of the same words, such that the terms mean that one or more of something is provided. The term “one” or “single” may be used to indicate that one and only one of something is intended. Similarly, other specific integer values, such as “two,” may be used when a specific number of things is intended. The terms “preferably,” “preferred,” “prefer,” “optionally,” “may,” and similar terms are used to indicate that an item, condition or step being referred to is an optional (not required) feature of the invention.

The invention has been described with reference to various specific and preferred embodiments and techniques. However, it should be understood that many variations and modifications may be made while remaining within the spirit and scope of the invention. It will be apparent to one of ordinary skill in the art that methods, devices, device elements, materials, procedures and techniques other than those specifically described herein can be applied to the practice of the invention as broadly disclosed herein without resort to undue experimentation. All art-known functional equivalents of methods, devices, device elements, materials, procedures and techniques described herein are intended to be encompassed by this invention. Whenever a range is disclosed, all sub-ranges and individual values are intended to be encompassed. This invention is not to be limited by the embodiments disclosed, including any shown in the drawings or exemplified in the specification, which are given by way of example and not of limitation. Additionally, it should be understood that the various embodiments of the suspension device described herein contain optional features that can be individually or together applied to any other embodiment shown or contemplated here to be mixed and matched with the features of that device.

While the invention has been described with respect to a limited number of embodiments, those skilled in the art, having benefit of this disclosure, will appreciate that other embodiments can be devised which do not depart from the scope of the invention as disclosed herein. Accordingly, the scope of the invention should be limited only by the attached claims.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

We claim:

1. A percutaneously inserted catheter device for delivering fluid around a vein comprising:
a. a housing at the distal end;
b. a heating element;
c. a needle to deliver fluid around the vein; and
d. a lumen for the needle to pass through.

2. The catheter device of claim 1 where the needle may have an end port for fluid delivery.

3. The catheter device of claim 1 where the needle may have bidirectional or multidirectional ports for fluid delivery.

4. The catheter device of claim 1 where the needle can be from 30 gauge to 14 gauge.

5. The catheter device of claim 1 where the needle may be echogenic to facilitate visibility on ultrasound.

6. The catheter device of claim 1 where the needle is made of nitinol.

7. The catheter device of claim 1 wherein the catheter includes a control mechanism to advance and retract the needle.

8. The catheter device of claim 1 where the projection of the needle beyond the lumen of the catheter and/or outside the vein can be controlled.

9. The catheter device of claim 1 where there is a control mechanism for advancing and retracting the needle.

10. The catheter device of claim 1 where there is an automatic retraction feature for the needle.

11. The catheter device of claim 1 where is there is an auto-stop advancing mechanism as a safety feature.

12. The catheter device of claim 1 where there is one, two, three or more lumens.

13. The catheter device of claim 1 where there is a balloon at varying lengths at or before the distal end.

14. The catheter device of claim 1 wherein the heating element is radiofrequency.

15. or laser or other forms or thermal energy generating devices can be inserted into it.

16. A method for ablating
a. inserted a catheter device through skin;
b. cannulating a vein and advancing the catheter device into the vein comprising a venous wall and peri-venous sheath;
c. advancing a needle through the venous wall and into the peri-venous sheath;
d. injecting tumescent around the vein through the needle;
e. ablating the vein segment that has been injected with tumescence; and;
f. drawing the catheter device a distance out of the vein.
